(19) United States
(12) Patent Application Publication
(10) Pub. No.: US 2026/0048072 A1
(43) Pub. Date: Feb. 19, 2026

(54) PHARMACEUTICAL PREPARATION FOR INTRANASAL ADMINISTRATION AND PREPARATION METHOD THEREFOR

(71) Applicant: CHENGDU EASTON BIOPHARMACEUTICALS CO., LTD., Chengdu (CN)

(72) Inventors: Yalan ZHANG, Chengdu (CN); Lingmei Guo, Chengdu (CN); Yan Chen, Chengdu (CN); Shuang Liu, Chengdu (CN); Jianjing Jia, Chengdu (CN); Yin Chen, Chengdu (CN); Xiuzhi Liu, Chengdu (CN); Yuhong He, Chengdu (CN); Hong Chen, Chengdu (CN); Ying Wang, Chengdu (CN)

(21) Appl. No.: 19/371,026

(22) Filed: Oct. 28, 2025

Related U.S. Application Data
(63) Continuation of application No. PCT/CN2024/090225, filed on Apr. 26, 2024.

(30) Foreign Application Priority Data
Apr. 28, 2023 (CN) 202310480177.5

(51) Int. Cl. A61K 31/685; A61K 9/00; A61K 31/485; A61K 47/02; A61K 47/12; A61K 47/18; A61K 47/26

(52) U.S. Cl. CPC A61K 31/685; A61K 9/0043; A61K 31/485; A61K 47/02; A61K 47/12; A61K 47/183; A61K 47/186; A61K 47/26

(57) ABSTRACT

The present invention relates to the field of pharmaceutical preparations, and in particular to a composition of a nalmefene hydrochloride nasal preparation and a preparation method therefor. The nalmefene hydrochloride nasal preparation comprises: nalmefene hydrochloride or a hydrate thereof and dodecylphosphorylcholine. A product of the nalmefene hydrochloride nasal preparation is absorbed by the nasal cavity rapidly, so that the problems of inconvenient administration and slow onset of existing preparations are solved, and the technical effect of providing safe, effective, and convenient clinical use is realized.

### CROSS-REFERENCE TO RELATED APPLICATIONS

This application is a continuation application of PCT Application No. PCT/CN2024/090225, filed on Apr. 26, 2024, which claims priority to Chinese Patent Application No. 202310480177.5, filed on Apr. 28, 2023. The entire disclosure of the applications identified in this paragraph are incorporated herein by references.

### TECHNICAL FIELD

The present invention belongs to the field of pharmaceutical preparations, and in particular to a stable nasal preparation of nalmefene hydrochloride, a preparation method and an administration method therefor.

### BACKGROUND

Nalmefene is an opioid antagonist that binds to the same brain opioid receptors as opioids, and has been used to antagonize respiratory depression caused by narcotic analgesics and in the treatment of heart failure, shock, alcohol intoxication, addiction, and the like. This antagonist not only can bind to the above receptors with high affinity, but also can compete with opioids for receptor sites, thereby reversing the pharmacological effects of opioids by displacing them from binding to the receptors.

Nalmefene hydrochloride is characterized by a long duration of action, high bioavailability, and few side effects. Although injections and oral preparations of nalmefene hydrochloride used clinically have certain therapeutic efficacy, oral administration has a slow onset of action, while intravenous and intramuscular injections require administration by professional medical personnel. For critically ill and comatose patients in need of timely and convenient administration at the earliest opportunity, as well as patients who experience sudden illness at home, it is essential to provide a route of administration other than injection and oral administration, or in other words, to provide an alternative route of administration.

Therefore, it is extremely necessary to provide a technical solution for a storage-stable, easy-to-use preparation and administration method that can enable untrained individuals to quickly deliver an effective dose of antagonists to patients with opioid overdose. Intranasal delivery is considered to be an attractive, safe, and easy-to-administer needle-free, systemic drug delivery route. In particular, when rapid absorption and onset of action are required, nasal administration has the advantages of rapid absorption, high bioavailability, and convenient administration, making it a hotspot in preparation research. To meet patients' needs for convenient administration, it is urgent to develop a nasal preparation of nalmefene hydrochloride that is easy to use in clinical and has a rapid onset of action.

### SUMMARY

Dodecylphosphocholine is a structural analogue of lauroyl lysophosphatidylcholine that is more stable to hydrolytic degradation, and is generally used as a detergent for the solubilization and purification of membrane proteins, with the following structure:

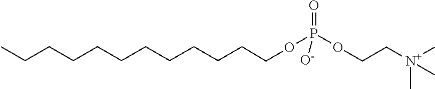 [SMILES: CCCCCCCCCCCCOP(=O)([O-])OCC[N+](C)(C)C]

The molecular weight of nalmefene hydrochloride=375.9.

The molecular weight of nalmefene free base=339.4.

In the pharmaceutical preparation of the present invention, the raw material used is nalmefene hydrochloride, and its amount calculated as nalmefene means that its content is calculated based on nalmefene free base. If converted to an amount calculated as nalmefene hydrochloride, it would be:

amount of nalmefene hydrochloride=375.9/339.4*amount of nalmefene.

The objective of the present invention is to provide a pharmaceutical preparation for intranasal administration, which, in about 50 μL to about 250 μL of an aqueous solution, comprises about 1 mg-10 mg of nalmefene hydrochloride, calculated as nalmefene, or the hydrate thereof, and dodecylphosphocholine. That is, it comprises 1.1 mg-11 mg of nalmefene hydrochloride or the hydrate thereof, calculated as nalmefene hydrochloride; and dodecylphosphocholine. Preferably, the intranasal administration preparation is a nasal spray preparation.

Preferably, the pharmaceutical preparation, in about 50 μL-250 μL of an aqueous solution, comprises about 2.5 mg-3.5 mg of nalmefene hydrochloride, calculated as nalmefene, or the hydrate thereof, and dodecylphosphocholine. That is, it comprises 2.8 mg-3.9 mg of nalmefene hydrochloride, or the hydrate thereof, calculated as nalmefene hydrochloride; and dodecylphosphocholine.

Preferably, the pharmaceutical preparation, in about 50 μL-250 μL of an aqueous solution, comprises about 2.7 mg-3.0 mg of nalmefene hydrochloride, calculated as nalmefene, or the hydrate thereof, and dodecylphosphocholine. That is, it comprises 3.0 mg-3.3 mg of nalmefene hydrochloride or the hydrate thereof, calculated as nalmefene hydrochloride; and dodecylphosphocholine.

Preferably, the pharmaceutical preparation comprises about 0.15 mg-0.5 mg of dodecylphosphocholine, and further preferably comprises about 0.15 mg-0.35 mg of dodecylphosphocholine.

Preferably, the pharmaceutical preparation comprises about 0.1 mg or 0.15 mg or 0.25 mg or 0.35 mg or 0.5 mg or 1 mg or 3 mg of dodecylphosphocholine.

Preferably, the pharmaceutical preparation comprises between about 1 mg and 10 mg of nalmefene hydrochloride, calculated as nalmefene, or the hydrate thereof; and about 0.01 mg-3 mg of dodecylphosphocholine.

Preferably, the pharmaceutical preparation comprises between about 2.5 mg and 3.5 mg of nalmefene hydrochloride, calculated as nalmefene, or the hydrate thereof; and about 0.01 mg-3 mg of dodecylphosphocholine.

Preferably, the pharmaceutical preparation comprises between about 2.7 mg and 3.0 mg of nalmefene hydrochloride, calculated as nalmefene, or the hydrate thereof; and about 0.01 mg-3 mg of dodecylphosphocholine.

Preferably, the pharmaceutical preparation comprises between about 2.7 mg and 3.0 mg of nalmefene hydrochloride, calculated as nalmefene, or the hydrate thereof; and about 0.15 mg-0.5 mg of dodecylphosphocholine.

Preferably, the pharmaceutical preparation comprises between about 2.7 mg and 3.0 mg of nalmefene hydrochloride, calculated as nalmefene, or the hydrate thereof; and about 0.15 mg-0.35 mg of dodecylphosphocholine.

Further preferably, the pharmaceutical preparation comprises about 3 mg of nalmefene hydrochloride, calculated as nalmefene, or the hydrate thereof; and about 0.01 mg-3 mg of dodecylphosphocholine.

Further preferably, the pharmaceutical preparation comprises about 3 mg of nalmefene hydrochloride, calculated as nalmefene, or the hydrate thereof; and about 0.01 mg-0.5 mg of dodecylphosphocholine.

Further preferably, the pharmaceutical preparation comprises about 3 mg of nalmefene hydrochloride, calculated as nalmefene, or the hydrate thereof; and about 0.01 mg-0.5 mg of dodecylphosphocholine; and one or more selected from a pH adjusting agent, a buffer, an osmotic pressure regulator, a preservative, or a stabilizer.

Further preferably, the pharmaceutical preparation comprises about 3 mg of nalmefene hydrochloride, calculated as nalmefene, or the hydrate thereof; and about 0.15 mg-0.5 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 3 mg of nalmefene hydrochloride, calculated as nalmefene, about 0.15 mg-0.5 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.35 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.5 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.35 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, or the hydrate thereof; and about 0.01 mg-3 mg of dodecylphosphocholine.

Further preferably, the pharmaceutical preparation comprises about 2.7 mg of nalmefene hydrochloride, calculated as nalmefene, or the hydrate thereof; and about 0.01 mg-0.5 mg of dodecylphosphocholine.

Further preferably, the pharmaceutical preparation comprises about 2.7 mg of nalmefene hydrochloride, calculated as nalmefene, or the hydrate thereof; and about 0.15 mg-0.5 mg of dodecylphosphocholine; and one or more selected from a pH adjusting agent, a buffer, an osmotic pressure regulator, a preservative, or a stabilizer.

Further preferably, the pharmaceutical preparation comprises about 2.7 mg of nalmefene hydrochloride, calculated as nalmefene, or the hydrate thereof; and about 0.15 mg-0.35 mg of dodecylphosphocholine; and one or more selected from a pH adjusting agent, a buffer, an osmotic pressure regulator, a preservative, or a stabilizer.

Further preferably, the pharmaceutical preparation comprises about 2.7 mg of nalmefene hydrochloride, calculated as nalmefene, or the hydrate thereof; and about 0.25 mg of dodecylphosphocholine; and one or more selected from a pH adjusting agent, a buffer, an osmotic pressure regulator, a preservative, or a stabilizer.

Further preferably, the pharmaceutical preparation comprises about 2.7 mg of nalmefene hydrochloride, calculated as nalmefene, or the hydrate thereof; and about 0.15 mg-0.5 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 2.7 mg of nalmefene hydrochloride, calculated as nalmefene, or the hydrate thereof; and about 0.15 mg-0.35 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 2.7 mg of nalmefene hydrochloride, calculated as nalmefene, about 0.15 mg-0.5 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, about 0.15 mg-0.35 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, about 0.15 mg-0.5 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5. Further preferably, the pharmaceutical preparation comprises about 2.7 mg of nalmefene hydrochloride, calculated as nalmefene, about 0.15 mg-0.35 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 0.25 mg of dodecylphosphocholine.

Further preferably, the pharmaceutical preparation comprises about 0.35 mg of dodecylphosphocholine.

Further preferably, it comprises a pH adjusting agent or a buffer to achieve a pH of 4.0.

Preferably, the pH adjusting agent or buffer is selected from one or more of hydrochloric acid, lactic acid, sodium hydroxide, acetic acid, sodium acetate, citric acid, sodium citrate, tartaric acid, sodium tartrate, adipic acid, potassium citrate, fumaric acid, sodium sesquicarbonate, malic acid, potassium carbonate, phosphoric acid, sodium carbonate, benzenesulfonic acid, benzoic acid, camphorsulfonic acid, ethylene sulfonic acid, dichloroacetic acid, formic acid, gluconic acid, glutamic acid, hippuric acid, hydrobromic acid, isethionic acid, maleic acid, mandelic acid, methanesulfonic acid, mucic acid, nitric acid, pamoic acid, pantothenic acid, succinic acid, sulfuric acid, oxalic acid, or p-toluenesulfonic acid.

Further preferably, the pH adjusting agent is hydrochloric acid and/or sodium hydroxide.

Further preferably, the buffer is a buffer pair of citric acid-sodium citrate.

Preferably, the osmotic pressure regulator is selected from one or more of glycerol, lactose, sodium chloride, potassium chloride, glucose, calcium chloride, magnesium chloride, sorbitol, sucrose, mannitol, trehalose, raffinose, polyethylene glycol, hydroxyethyl starch, glycine, and dextran.

Further preferably, the osmotic pressure regulator is sodium chloride.

Further preferably, it comprises about 0.74 mg of sodium chloride.

Preferably, the preservative is selected from one or more of propylene glycol, benzalkonium chloride, benzalkonium bromide, methyl paraben, ethyl paraben, propyl paraben, butyl paraben, benzoic acid, sodium benzoate, phenethyl alcohol, phenol, cresol, chlorocresol, chlorobutanol, benzyl alcohol, and thimerosal.

Further preferably, the preservative is benzalkonium chloride.

Further preferably, it comprises about 0.01 mg of benzalkonium chloride.

Preferably, the stabilizer is selected from one or more of edetate disodium, calcium sodium edetate, sodium pyrosulfite, sodium thiosulfate, sodium sulfite, vitamin C, and cysteine hydrochloride.

Further preferably, the stabilizer is edetate disodium.

Further preferably, it comprises about 0.1 mg of edetate disodium.

Preferably, the pharmaceutical preparation is about 100 μL of an aqueous solution.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.15 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5. Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.35 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.15 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.35 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.25 mg of dodecylphosphocholine; and a buffer pair of citric acid-sodium citrate to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and a buffer pair of citric acid-sodium citrate to achieve a pH of 3.5-5.5.

Preferably, the present invention provides a pharmaceutical preparation for intranasal administration, which, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and about 0.1 mg of edetate disodium; and a buffer pair of citric acid-sodium citrate to achieve a pH of 3.5-5.5.

Another objective of the present invention is to provide a pharmaceutical preparation for intranasal administration, which, in about 50 μL-250 μL of an aqueous solution, comprises between about 1% and 10% (w/v) of nalmefene hydrochloride, calculated as nalmefene, or the hydrate thereof, and dodecylphosphocholine. Preferably, the intranasal administration preparation is a nasal spray preparation.

Preferably, the pharmaceutical preparation comprises between about 1% and 10% (w/v) of nalmefene hydrochloride, calculated as nalmefene, or the hydrate thereof; and about 0.01%-3% (w/v) of dodecylphosphocholine. That is, it comprises 1.1%-11% (w/v) of nalmefene hydrochloride or the hydrate thereof, calculated as nalmefene hydrochloride; and about 0.01%-3% (w/v) of dodecylphosphocholine.

Preferably, the pharmaceutical preparation comprises between about 2.5% and 3.5% (w/v) of nalmefene hydrochloride, calculated as nalmefene, or the hydrate thereof; and about 0.01%-3% (w/v) of dodecylphosphocholine. That is, it comprises 2.8%-3.9% (w/v) of nalmefene hydrochloride or the hydrate thereof, calculated as nalmefene hydrochloride; and about 0.01%-3% (w/v) of dodecylphosphocholine.

Preferably, the pharmaceutical preparation comprises between about 2.7% and 3.0% (w/v) of nalmefene hydrochloride, calculated as nalmefene, or the hydrate thereof; and about 0.01%-3% (w/v) of dodecylphosphocholine. That is, it comprises 3.0%-3.3% (w/v) of nalmefene hydrochloride or the hydrate thereof, calculated as nalmefene hydrochloride; and about 0.01%-3% (w/v) of dodecylphosphocholine.

Preferably, the pharmaceutical preparation comprises about 0.15%-0.5% (w/v) of dodecylphosphocholine, and further preferably comprises about 0.15%-0.35% (w/v) of dodecylphosphocholine.

Further preferably, the pharmaceutical preparation comprises about 0.1% (w/v) or 0.15% (w/v) or 0.25% (w/v) or 0.35% (w/v) or 0.5% (w/v) or 1% (w/v) or 3% (w/v) of dodecylphosphocholine.

Preferably, the pharmaceutical preparation comprises between about 2.7% and 3.0% (w/v) of nalmefene hydrochloride, calculated as nalmefene, or the hydrate thereof; and about 0.15%-0.35% (w/v) of dodecylphosphocholine.

Further preferably, the pharmaceutical preparation comprises about 3% (w/v) of nalmefene hydrochloride, calculated as nalmefene, or the hydrate thereof; and about 0.01%-3% (w/v) of dodecylphosphocholine.

Further preferably, the pharmaceutical preparation comprises about 3% (w/v) of nalmefene hydrochloride, calculated as nalmefene, or the hydrate thereof; and about 0.15%-0.5% (w/v) of dodecylphosphocholine.

Further preferably, the pharmaceutical preparation comprises about 3% (w/v) of nalmefene hydrochloride, calculated as nalmefene, or the hydrate thereof; and about 0.15%-0.35% (w/v) of dodecylphosphocholine; and one or more selected from a pH adjusting agent, a buffer, an osmotic pressure regulator, a preservative, or a stabilizer.

Further preferably, the pharmaceutical preparation comprises about 3% (w/v) of nalmefene hydrochloride, calculated as nalmefene, or the hydrate thereof; and about 0.25% (w/v) of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001%-1% (w/v) of a preservative, or about 0.001%-1% (w/v) of a stabilizer.

Further preferably, the pharmaceutical preparation comprises about 3% (w/v) of nalmefene hydrochloride, calculated as nalmefene, about 0.15%-0.5% (w/v) of dodecylphosphocholine, about 0.74% (w/v) of sodium chloride, and hydrochloric acid and/or sodium hydroxide to achieve a pH of 3.5-5.5.

Further preferably, the pharmaceutical preparation comprises about 3% (w/v) of nalmefene hydrochloride, calculated as nalmefene, about 0.15%-0.35% (w/v) of dodecylphosphocholine, about 0.74% (w/v) of sodium chloride, and hydrochloric acid and/or sodium hydroxide to achieve a pH of 3.5-5.5.

Further preferably, the pharmaceutical preparation comprises about 3% (w/v) of nalmefene hydrochloride, calculated as nalmefene, about 0.15%-0.5% (w/v) of dodecylphosphocholine, about 0.74% (w/v) of sodium chloride, about 0.1% (w/v) edetate disodium, about 0.01% (w/v) benzalkonium chloride, and hydrochloric acid and/or sodium hydroxide to achieve a pH of 3.5-5.5.

Further preferably, the pharmaceutical preparation comprises about 3% (w/v) of nalmefene hydrochloride, calculated as nalmefene, about 0.15%-0.35% (w/v) of dodecylphosphocholine, about 0.74% (w/v) of sodium chloride, about 0.1% (w/v) edetate disodium, about 0.01% (w/v) benzalkonium chloride, and hydrochloric acid and/or sodium hydroxide to achieve a pH of 3.5-5.5.

Further preferably, the pharmaceutical preparation comprises about 2.7% (w/v) of nalmefene hydrochloride, calculated as nalmefene, or the hydrate thereof; and about 0.01%-3% (w/v) of dodecylphosphocholine.

Further preferably, the pharmaceutical preparation comprises about 2.7% (w/v) of nalmefene hydrochloride, calculated as nalmefene, or the hydrate thereof; and about 0.01%-0.5% (w/v) of dodecylphosphocholine.

Further preferably, the pharmaceutical preparation comprises about 2.7% (w/v) of nalmefene hydrochloride, calculated as nalmefene, or the hydrate thereof; and about 0.15%-0.5% (w/v) of dodecylphosphocholine.

Further preferably, the pharmaceutical preparation comprises about 2.7% (w/v) of nalmefene hydrochloride, calculated as nalmefene, or the hydrate thereof; and about 0.15%-0.35% (w/v) of dodecylphosphocholine.

Further preferably, the pharmaceutical preparation comprises about 2.7% (w/v) of nalmefene hydrochloride, calculated as nalmefene, or the hydrate thereof; and about 0.25% (w/v) of dodecylphosphocholine.

Further preferably, the pharmaceutical preparation comprises about 2.7% (w/v) of nalmefene hydrochloride, calculated as nalmefene, or the hydrate thereof; and about 0.01%-0.5% (w/v) of dodecylphosphocholine; and one or more selected from a pH adjusting agent, a buffer, an osmotic pressure regulator, a preservative, or a stabilizer.

Further preferably, the pharmaceutical preparation comprises about 2.7% (w/v) of nalmefene hydrochloride, calculated as nalmefene, or the hydrate thereof; and about 0.15%-0.5% (w/v) of dodecylphosphocholine; and one or more selected from a pH adjusting agent, a buffer, an osmotic pressure regulator, a preservative, or a stabilizer.

Further preferably, the pharmaceutical preparation comprises about 2.7% (w/v) of nalmefene hydrochloride, calculated as nalmefene, or the hydrate thereof; and about 0.15%-0.35% (w/v) of dodecylphosphocholine; and one or more selected from a pH adjusting agent, a buffer, an osmotic pressure regulator, a preservative, or a stabilizer.

Further preferably, the pharmaceutical preparation comprises about 2.7% (w/v) of nalmefene hydrochloride, calculated as nalmefene, or the hydrate thereof; and about 0.15%-0.5% (w/v) of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001%-1% (w/v) of a preservative, or about 0.001%-1% (w/v) of a stabilizer.

Further preferably, the pharmaceutical preparation comprises about 2.7% (w/v) of nalmefene hydrochloride, calculated as nalmefene, or the hydrate thereof; and about 0.15%-0.35% (w/v) of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001%-1% (w/v) of a preservative, or about 0.001%-1% (w/v) of a stabilizer.

Further preferably, the pharmaceutical preparation comprises about 2.7% (w/v) of nalmefene hydrochloride, calculated as nalmefene, or the hydrate thereof; and about 0.25%-0.35% (w/v) of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001%-1% (w/v) of a preservative, or about 0.001%-1% (w/v) of a stabilizer.

Further preferably, the pharmaceutical preparation comprises about 2.7% (w/v) of nalmefene hydrochloride, calculated as nalmefene, about 0.15%-0.5% (w/v) of dodecylphosphocholine, about 0.74% (w/v) of sodium chloride, and hydrochloric acid and/or sodium hydroxide to achieve a pH of 3.5-5.5.

Further preferably, the pharmaceutical preparation comprises about 2.7% (w/v) of nalmefene hydrochloride, calculated as nalmefene, about 0.15%-0.35% (w/v) of dodecylphosphocholine, about 0.74% (w/v) of sodium chloride, and hydrochloric acid and/or sodium hydroxide to achieve a pH of 3.5-5.5.

Further preferably, the pharmaceutical preparation comprises about 2.7% (w/v) of nalmefene hydrochloride, calculated as nalmefene, about 0.15%-0.5% (w/v) of dodecylphosphocholine, about 0.74% (w/v) of sodium chloride, about 0.1% (w/v) edetate disodium, about 0.01% (w/v) benzalkonium chloride, and hydrochloric acid and/or sodium hydroxide to achieve a pH of 3.5-5.5.

Further preferably, the pharmaceutical preparation comprises about 2.7% (w/v) of nalmefene hydrochloride, calculated as nalmefene, about 0.15%-0.35% (w/v) of dodecylphosphocholine, about 0.74% (w/v) of sodium chloride, about 0.1% (w/v) edetate disodium, about 0.01% (w/v) benzalkonium chloride, and hydrochloric acid and/or sodium hydroxide to achieve a pH of 3.5-5.5.

Further preferably, the pharmaceutical preparation comprises about 0.25% (w/v) of dodecylphosphocholine.

Further preferably, the pharmaceutical preparation comprises about 0.35% (w/v) of dodecylphosphocholine.

Further preferably, it comprises a pH adjusting agent or a buffer to achieve a pH of 4.0.

Preferably, the pH adjusting agent or buffer is selected from one or more of hydrochloric acid, lactic acid, sodium hydroxide, acetic acid, sodium acetate, citric acid, sodium citrate, tartaric acid, sodium tartrate, adipic acid, potassium citrate, fumaric acid, sodium sesquicarbonate, malic acid, potassium carbonate, phosphoric acid, sodium carbonate, benzenesulfonic acid, benzoic acid, camphorsulfonic acid, ethylene sulfonic acid, dichloroacetic acid, formic acid, gluconic acid, glutamic acid, hippuric acid, hydrobromic acid, isethionic acid, maleic acid, mandelic acid, methanesulfonic acid, mucic acid, nitric acid, pamoic acid, pantothenic acid, succinic acid, sulfuric acid, oxalic acid, or p-toluenesulfonic acid.

Further preferably, the pH adjusting agent is hydrochloric acid and/or sodium hydroxide.

Further preferably, the buffer is a buffer pair of citric acid-sodium citrate.

Preferably, the osmotic pressure regulator is selected from one or more of glycerol, lactose, sodium chloride, potassium chloride, glucose, calcium chloride, magnesium chloride, sorbitol, sucrose, mannitol, trehalose, raffinose, polyethylene glycol, hydroxyethyl starch, glycine, and dextran.

Further preferably, the osmotic pressure regulator is sodium chloride.

Further preferably, it comprises about 0.74% (w/v) of sodium chloride.

Preferably, the preservative is selected from one or more of propylene glycol, benzalkonium chloride, benzalkonium bromide, methyl paraben, ethyl paraben, propyl paraben, butyl paraben, benzoic acid, sodium benzoate, phenethyl alcohol, phenol, cresol, chlorocresol, chlorobutanol, benzyl alcohol, and thimerosal.

Further preferably, the preservative is benzalkonium chloride.

Further preferably, it comprises about 0.01% (w/v) of benzalkonium chloride.

Preferably, the stabilizer is selected from one or more of edetate disodium, calcium sodium edetate, sodium pyrosulfite, sodium thiosulfate, sodium sulfite, vitamin C, and cysteine hydrochloride.

Further preferably, the stabilizer is edetate disodium.

Further preferably, it comprises about 0.1% (w/v) of edetate disodium.

Preferably, the pharmaceutical preparation is about 100 μL of an aqueous solution.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.25% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.5% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.25% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.5% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.15% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.25% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.35% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.5% (w/v) of dodecylphosphocholine, and about 0.74% (w/v) of sodium chloride; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.15% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.25% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.35% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 2.7% (w/v) of nalmefene hydrochloride, calculated as nalmefene, or the hydrate thereof; and about 0.5% (w/v) of dodecylphosphocholine; and about 0.74% (w/v) of sodium chloride; and about 0.01% (w/v) of benzalkonium chloride; and about 0.1% (w/v) of edetate disodium; and hydrochloric acid and/or sodium hydroxide to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.25% (w/v) of dodecylphosphocholine; and a buffer pair of citric acid-sodium citrate to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.25% (w/v) of dodecylphosphocholine; and about 0.01% (w/v) of benzalkonium chloride; and a buffer pair of citric acid-sodium citrate to achieve a pH of 3.5-5.5.

Preferably, the present invention provides a pharmaceutical preparation for intranasal administration, which, in about 100 μL of an aqueous solution, comprises about 3% (w/v) of nalmefene hydrochloride, calculated as nalmefene, or the hydrate thereof; and about 0.25% (w/v) of dodecylphosphocholine; and about 0.01% (w/v) of benzalkonium chloride; and about 0.1% (w/v) of edetate disodium; and a buffer pair of citric acid-sodium citrate to achieve a pH of 3.5-5.5.

Another objective of the present invention is to provide a pharmaceutical preparation for nasal spray, which, in about 50 μL-250 μL of an aqueous solution, comprises about 1 mg-10 mg of nalmefene hydrochloride, calculated as nalmefene, or the hydrate thereof, and dodecylphosphocholine. That is, it comprises 1.1 mg-11 mg of nalmefene hydrochloride or the hydrate thereof, calculated as nalmefene hydrochloride; and dodecylphosphocholine.

Preferably, the pharmaceutical preparation comprises between about 2.5 mg and 3.5 mg of nalmefene hydrochloride, calculated as nalmefene, or the hydrate thereof; and dodecylphosphocholine. That is, it comprises 2.8 mg-3.9 mg of nalmefene hydrochloride or the hydrate thereof, calculated as nalmefene hydrochloride; and dodecylphosphocholine.

Preferably, the pharmaceutical preparation comprises between about 2.7 mg and 3.0 mg of nalmefene hydrochloride, calculated as nalmefene, or the hydrate thereof; and dodecylphosphocholine. That is, it comprises 3.0 mg-3.3 mg of nalmefene hydrochloride or the hydrate thereof, calculated as nalmefene hydrochloride; and dodecylphosphocholine. Preferably, the pharmaceutical preparation comprises between about 1 mg and 10 mg of nalmefene hydrochloride, calculated as nalmefene, or the hydrate thereof; and about 0.01 mg-3 mg of dodecylphosphocholine.

Preferably, the pharmaceutical preparation comprises between about 2.5 mg and 3.5 mg of nalmefene hydrochloride, calculated as nalmefene, or the hydrate thereof; and about 0.01 mg-3 mg of dodecylphosphocholine.

Preferably, the pharmaceutical preparation comprises between about 2.7 mg and 3.0 mg of nalmefene hydrochloride, calculated as nalmefene, or the hydrate thereof; and about 0.01 mg-3 mg of dodecylphosphocholine.

Preferably, the pharmaceutical preparation comprises about 0.15 mg-0.5 mg of dodecylphosphocholine, and further preferably comprises about 0.15 mg-0.35 mg of dodecylphosphocholine.

Preferably, the pharmaceutical preparation comprises about 0.1 mg or 0.15 mg or 0.25 mg or 0.35 mg or 0.5 mg or 1 mg or 3 mg of dodecylphosphocholine.

Further preferably, the pharmaceutical preparation comprises about 3 mg of nalmefene hydrochloride, calculated as nalmefene, or the hydrate thereof; and about 0.01 mg-3 mg of dodecylphosphocholine.

Further preferably, the pharmaceutical preparation comprises about 3 mg of nalmefene hydrochloride, calculated as nalmefene, or the hydrate thereof; and about 0.15 mg-0.5 mg of dodecylphosphocholine.

Further preferably, the pharmaceutical preparation comprises about 3 mg of nalmefene hydrochloride, calculated as nalmefene, or the hydrate thereof; and about 0.15 mg-0.35 mg of dodecylphosphocholine; and one or more selected from a pH adjusting agent, a buffer, an osmotic pressure regulator, a preservative, or a stabilizer.

Further preferably, the pharmaceutical preparation comprises about 3 mg of nalmefene hydrochloride, calculated as nalmefene, or the hydrate thereof; and about 0.15 mg-0.5 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 3 mg of nalmefene hydrochloride, calculated as nalmefene, about 0.15 mg-0.5 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.35 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.5 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 3 mg of nalmefene hydrochloride, calculated as nalmefene, about 0.15 mg-0.35 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, or the hydrate thereof; and about 0.01 mg-0.5 mg of dodecylphosphocholine.

Further preferably, the pharmaceutical preparation comprises about 2.7 mg of nalmefene hydrochloride, calculated as nalmefene, or the hydrate thereof; and about 0.01 mg-3 mg of dodecylphosphocholine.

Further preferably, the pharmaceutical preparation comprises about 2.7 mg of nalmefene hydrochloride, calculated as nalmefene, or the hydrate thereof; and about 0.15 mg-0.5 mg of dodecylphosphocholine.

Further preferably, the pharmaceutical preparation comprises about 2.7 mg of nalmefene hydrochloride, calculated as nalmefene, or the hydrate thereof; and about 0.15 mg-0.35 mg of dodecylphosphocholine.

Further preferably, the pharmaceutical preparation comprises about 2.7 mg of nalmefene hydrochloride, calculated as nalmefene, or the hydrate thereof; and about 0.25 mg-0.35 mg of dodecylphosphocholine; and one or more selected from a pH adjusting agent, a buffer, an osmotic pressure regulator, a preservative, or a stabilizer.

Further preferably, the pharmaceutical preparation comprises about 2.7 mg of nalmefene hydrochloride, calculated as nalmefene, or the hydrate thereof; and about 0.01 mg-0.5 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 2.7 mg of nalmefene hydrochloride, calculated as nalmefene, or the hydrate thereof; and about 0.15 mg-0.35 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation comprises about 2.7 mg of nalmefene hydrochloride, calculated as nalmefene, about 0.15 mg-0.5 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, about 0.15 mg-0.35 mg of dodecylphosphocholine, about 0.74 mg of sodium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, about 0.15 mg-0.5 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 2.7 mg of nalmefene hydrochloride, calculated as nalmefene, about 0.15 mg-0.35 mg of dodecylphosphocholine, about 0.74 mg of sodium chloride, about 0.1 mg of edetate disodium, about 0.01 mg of benzalkonium chloride, and hydrochloric acid and/or sodium hydroxide to achieve a pH of 3.5-5.5.

Further preferably, the pharmaceutical preparation comprises about 0.25 mg of dodecylphosphocholine.

Further preferably, the pharmaceutical preparation comprises about 0.35 mg of dodecylphosphocholine.

Preferably, the pharmaceutical preparation, in about 100 μL of an aqueous solution, comprises about 1 mg-10 mg of nalmefene hydrochloride, calculated as nalmefene, or the hydrate thereof; and dodecylphosphocholine.

Preferably, the pharmaceutical preparation, in about 100 μL of an aqueous solution, comprises about 2.5 mg-3.5 mg of nalmefene hydrochloride, calculated as nalmefene, or the hydrate thereof; and dodecylphosphocholine.

Preferably, the pharmaceutical preparation, in about 100 μL of an aqueous solution, comprises about 2.7 mg-3.0 mg of nalmefene hydrochloride, calculated as nalmefene, or the hydrate thereof; and dodecylphosphocholine.

Further preferably, the pharmaceutical preparation, in about 100 μL of an aqueous solution, comprises about 1 mg-10 mg of nalmefene hydrochloride, calculated as nalmefene, or the hydrate thereof; and about 0.01 mg-3 mg of dodecylphosphocholine.

Further preferably, the pharmaceutical preparation, in about 100 μL of an aqueous solution, comprises about 2.5 mg-3.5 mg of nalmefene hydrochloride, calculated as nalmefene, or the hydrate thereof; and about 0.01 mg-3 mg of dodecylphosphocholine.

Further preferably, the pharmaceutical preparation, in about 100 μL of an aqueous solution, comprises about 2.7 mg-3.0 mg of nalmefene hydrochloride, calculated as nalmefene, or the hydrate thereof; and about 0.01 mg-3 mg of dodecylphosphocholine.

Further preferably, the pharmaceutical preparation, in about 100 μL of an aqueous solution, comprises about 3 mg of nalmefene hydrochloride, calculated as nalmefene, or the hydrate thereof; and about 0.01 mg-3 mg of dodecylphosphocholine.

Further preferably, the pharmaceutical preparation, in about 100 μL of an aqueous solution, comprises about 2.7 mg of nalmefene hydrochloride, calculated as nalmefene, or the hydrate thereof; and about 0.01 mg-3 mg of dodecylphosphocholine.

Preferably, the pharmaceutical preparation comprises about 0.15 mg-0.5 mg of dodecylphosphocholine, and further preferably comprises about 0.15 mg-0.35 mg of dodecylphosphocholine.

Preferably, the pharmaceutical preparation comprises about 0.1 mg or 0.15 mg or 0.25 mg or 0.35 mg or 0.5 mg or 1 mg or 3 mg of dodecylphosphocholine.

Further preferably, the pharmaceutical preparation, in about 100 μL of an aqueous solution, comprises about 3 mg of nalmefene hydrochloride, calculated as nalmefene, or the hydrate thereof; and about 0.01 mg-0.5 mg of dodecylphosphocholine.

Further preferably, the pharmaceutical preparation, in about 100 μL of an aqueous solution, comprises about 3 mg of nalmefene hydrochloride, calculated as nalmefene, or the hydrate thereof; and about 0.15 mg-0.5 mg of dodecylphosphocholine; and one or more selected from a pH adjusting agent, a buffer, an osmotic pressure regulator, a preservative, or a stabilizer.

Further preferably, the pharmaceutical preparation, in about 100 μL of an aqueous solution, comprises about 3 mg of nalmefene hydrochloride, calculated as nalmefene, or the hydrate thereof; and about 0.15 mg-0.35 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Further preferably, the pharmaceutical preparation, in about 100 μL of an aqueous solution, comprises about 2.7 mg of nalmefene hydrochloride, calculated as nalmefene, or the hydrate thereof; and about 0.01 mg-0.5 mg of dodecylphosphocholine.

Further preferably, the pharmaceutical preparation, in about 100 μL of an aqueous solution, comprises about 2.7 mg of nalmefene hydrochloride, calculated as nalmefene, or the hydrate thereof; and about 0.15 mg-0.5 mg of dodecylphosphocholine; and one or more selected from a pH adjusting agent, a buffer, an osmotic pressure regulator, a preservative, or a stabilizer.

Further preferably, the pharmaceutical preparation, in about 100 μL of an aqueous solution, comprises about 2.7 mg of nalmefene hydrochloride, calculated as nalmefene, or the hydrate thereof; and about 0.15 mg-0.35 mg of dodecylphosphocholine; and one or more selected from a pH adjusting agent, a buffer, an osmotic pressure regulator, a preservative, or a stabilizer.

Further preferably, the pharmaceutical preparation, in about 100 μL of an aqueous solution, comprises about 2.7 mg of nalmefene hydrochloride, calculated as nalmefene, or the hydrate thereof; and about 0.15 mg-0.5 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Further preferably, the pharmaceutical preparation, in about 100 μL of an aqueous solution, comprises about 2.7 mg of nalmefene hydrochloride, calculated as nalmefene, or the hydrate thereof; and about 0.15 mg-0.35 mg of dodecylphosphocholine; and one or more selected from a pH adjusting agent or a buffer to achieve a pH of about 3.5-5.5, an osmotic pressure regulator to achieve an osmotic pressure ratio of about 0.2-2, about 0.001 mg-1 mg of a preservative, or about 0.001 mg-1 mg of a stabilizer.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.15 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.35 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.15 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.35 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 2.7 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.25 mg of dodecylphosphocholine; and a buffer pair of citric acid-sodium citrate to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and a buffer pair of citric acid-sodium citrate to achieve a pH of 3.5-5.5.

Preferably, the present invention provides a pharmaceutical preparation for nasal spray, which, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and about 0.1 mg of edetate disodium; and a buffer pair of citric acid-sodium citrate to achieve a pH of 3.5-5.5.

Another objective of the present invention is to provide a method for preventing or treating opioid overdose or the symptoms thereof in patients, by providing a therapeutically effective amount of a nasal preparation of nalmefene hydrochloride or the hydrate thereof, to individuals at risk of opioid overdose, wherein, the nasal preparation comprises about 3 mg of nalmefene hydrochloride, calculated as nalmefene, or the hydrate thereof; and about 0.15 mg-0.5 mg of dodecylphosphocholine, and one or more selected from a pH adjusting agent, a buffer, an osmotic pressure regulator, a preservative, and a stabilizer.

Preferably, the nasal preparation, in about 100 μL of an aqueous solution, comprises about 3 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.25 mg of dodecylphosphocholine; and a buffer pair of citric acid-sodium citrate to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and a buffer pair of citric acid-sodium citrate to achieve a pH of 3.5-5.5.

Preferably, the nasal preparation, in about 100 μL of an aqueous solution, comprises about 3 mg of nalmefene hydrochloride, calculated as nalmefene, or the hydrate thereof; and about 0.25 mg of dodecylphosphocholine; and about 0.01 mg of benzalkonium chloride; and about 0.1 mg of edetate disodium; and a buffer pair of citric acid-sodium citrate to achieve a pH of 3.5-5.5.

Another objective of the present invention is to provide a method for preventing or treating opioid overdose or the symptoms thereof in patients, by providing a therapeutically effective amount of a nasal preparation of nalmefene hydrochloride or the hydrate thereof, to individuals at risk of opioid overdose, wherein, the nasal preparation comprises about 2.7 mg of nalmefene hydrochloride, calculated as nalmefene, or the hydrate thereof; and about 0.15 mg-0.5 mg of dodecylphosphocholine, and one or more selected from a pH adjusting agent, a buffer, an osmotic pressure regulator, a preservative, and a stabilizer.

Preferably, the nasal preparation, in about 100 μL of an aqueous solution, comprises about 2.7 mg of nalmefene hydrochloride, calculated as nalmefene, or the hydrate thereof; and about 0.15 mg-0.5 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.15 mg-0.35 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.25 mg-0.35 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.15 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.25 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.35 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.5 mg of dodecylphosphocholine, and about 0.74 mg of sodium chloride; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.15 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.25 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.35 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

Preferably, the nasal preparation, in about 100 μL of an aqueous solution, comprises about 2.7 mg of nalmefene hydrochloride, calculated as nalmefene, or the hydrate thereof; and about 0.5 mg of dodecylphosphocholine; and about 0.74 mg of sodium chloride; and about 0.01 mg of benzalkonium chloride; and about 0.1 mg of edetate disodium; and hydrochloric acid and/or sodium hydroxide to achieve a pH of 3.5-5.5.

The present invention utilizes dodecylphosphocholine as an absorption enhancer, and provides a nasal administration preparation of nalmefene that is easy to use in clinical and has a rapid onset of action. The absorption promoter can significantly accelerate the absorption of nalmefene hydrochloride in the nasal cavity, and the onset of action after administration is faster than that of intramuscular injection of 1.5 mg of a nalmefene hydrochloride injection. The present invention provides an improved nasal spray preparation of nalmefene hydrochloride that utilizes dodecylphosphocholine as an absorption enhancer to enhance its absorption-promoting effect. The absorption-promoting effect of dodecylphosphocholine in the nasal administration preparation of nalmefene hydrochloride is superior to that of the absorption promoter n-dodecyl-β-D-maltoside in the prior art. The nasal administration preparation provided by the present invention is superior to the commercially available product OPVEE in terms of both quality and stability.

### BRIEF DESCRIPTION OF THE DRAWINGS

FIG. 1 is a comparative diagram of the PK data of Examples 1-2 and Comparative Examples 1-2 of the present invention.

### DETAILED DESCRIPTION

Hereinafter, further detailed description of the present invention in conjunction with the Examples is made, but is not intended to limit the present invention. Any equivalent substitutions made in the art according to the disclosure of the present invention all belong to the scope of protection of the present invention.

The present invention discloses compositions and methods for treating opioid overdose, comprising administering an intranasal preparation of an opioid antagonist. For clarity and consistency, the following definitions are used in the present patent document.

The term “about” is intended to define the numerical value it modifies, indicating that such a value varies within a certain range. When a range, such as a margin of error or the standard deviation of a mean value given in a data diagram or table, is not listed, the term “about” should be understood to mean a broader range that includes the range of the recited value, a range included by rounding up or down to that digit in consideration of significant figures, and a range encompassing the recited value plus or minus 20%, preferably ±5%, and more preferably ±3%.

The term “agonist” refers to a substance that binds to and activates a receptor, thereby triggering the physiological or pharmacological response characteristics of that receptor; the term “antagonist” refers to a substance that competitively binds to the same receptor site as an agonist but does not activate the cellular response elicited by the active receptor, thereby inhibiting the intracellular response through the agonist or partial agonist.

The term “hydrate” refers to nalmefene or its hydrochloride described herein that further includes a stoichiometric or non-stoichiometric amount of water bound through non-covalent intermolecular forces.

### Example 1 Formulation and Preparation Thereof

The formulation examples of nalmefene disclosed in the present invention and the comparative examples are listed in Table 1-1 and Table 1-2.

TABLE 1-1

| | Example | Example | Example | Example | Example | Example |
|---|---|---|---|---|---|---|
| Component | 1 | 2 | 3 | 4 | 5 | 6 |
| Nalmefene | 3 | 3 | 3 | 3 | 3 | 3 |
| hydrochloride | | | | | | |
| (calculated | | | | | | |
| as nalmefene) | | | | | | |
| Dodecylphospho- | 0.25 | 0.5 | 0.25 | 0.5 | 0.25 | 0.25 |
| choline | | | | | | |
| n-Dodecyl-β-D- | NA | NA | NA | NA | NA | NA |
| maltoside | | | | | | |
| Sodium chloride | 0.74 | 0.74 | 0.74 | 0.74 | NA | NA |
| Anhydrous | NA | NA | NA | NA | 1.36 | 1.36 |
| citric acid | | | | | | |
| Sodium citrate | NA | NA | NA | NA | 1.50 | 1.50 |
| Hydrochloric | an | an | an | an | NA | NA |
| acid/Sodium | appropriate | appropriate | appropriate | appropriate | | |
| hydroxide | amount | amount | amount | amount | | |
| Benzal- | NA | NA | 0.01 | 0.01 | NA | 0.01 |
| konium | | | | | | |
| chloride | | | | | | |
| Edetate disodium | NA | NA | 0.1 | 0.1 | NA | NA |
| Water | q.s. to | q.s. to | q.s. to | q.s. to | q.s. to | q.s. to |
| | 0.1 mL | 0.1 mL | 0.1 mL | 0.1 mL | 0.1 mL | 0.1 mL |

| | Example | Example | Example | Example | Comparative |
|---|---|---|---|---|---|
| Component | 7 | 8 | 9 | 10 | Example 1 |
| Nalmefene | 3 | 3 | 2.7 | 2.7 | 3 |
| hydrochloride | | | | | |
| (calculated | | | | | |
| as nalmefene) | | | | | |
| Dodecylphospho- | 0.25 | 0.15 | 0.25 | 0.35 | NA |
| choline | | | | | |
| n-Dodecyl-β-D- | NA | NA | NA | NA | NA |
| maltoside | | | | | |
| Sodium chloride | NA | 0.74 | 0.74 | 0.74 | 0.74 |
| Anhydrous | 1.36 | NA | NA | NA | NA |
| citric acid | | | | | |
| Sodium citrate | 1.50 | NA | NA | NA | NA |
| Hydrochloric | NA | an | an | an | an |
| acid/Sodium | | appropriate | appropriate | appropriate | appropriate |
| hydroxide | | amount | amount | amount | amount |
| Benzal- | 0.01 | 0.01 | 0.01 | 0.01 | NA |
| konium | | | | | |
| chloride | | | | | |
| Edetate disodium | 0.1 | 0.1 | 0.1 | 0.1 | NA |
| Water | q.s. to | q.s. to | q.s. to | q.s. to | q.s. to |
| | 0.1 mL | 0.1 mL | 0.1 mL | 0.1 mL | 0.1 mL |

| | Comparative | Comparative | Comparative | Comparative | Comparative | Comparative |
|---|---|---|---|---|---|---|
| Component | Example 2 | Example 3 | Example 4 | Example 5 | Example 6 | Example 7 |
| Nalmefene | 1.5 | 3 | 3 | 2.7 | 2.7 | Commercially |
| hydrochloride | | | | | | available product |
| (calculated as | | | | | | OPVEE ® |
| nalmefene) | | | | | | purchased, batch |
| Dodecylphospho- | NA | NA | NA | NA | NA | No.: VNPA2305, |
| choline | | | | | | supplier: Indivior |
| n-Dodecyl-β-D- | NA | 0.25 | 0.25 | 0.25 | 0.25 | Inc. |
| maltoside | | | | | | |
| Sodium chloride | 13.5 | 0.74 | 0.74 | 0.90 | 0.74 | |
| Anhydrous | NA | NA | NA | NA | NA | |
| citric acid | | | | | | |
| Sodium citrate | NA | NA | NA | NA | NA | |
| Hydrochloric | an | an | an | NA | an | |
| acid/Sodium | appropriate | appropriate | appropriate | | appropriate | |
| hydroxide | amount | amount | amount | | amount | |
| Benzal- | NA | NA | 0.01 | 0.02 | 0.01 | |
| konium | | | | | | |
| chloride | | | | | | |
| Edetate disodium | NA | NA | 0.1 | 0.2 | 0.1 | |
| Water | q.s. to | q.s. to | q.s. to | q.s. to | q.s. to | |
| | 1.5 mL | 0.1 mL | 0.1 mL | 0.1 mL | 0.1 mL | |

Note:
The unit of mass is mg; Comparative Example 2 is an injection, administered intramuscularly.

TABLE 1-2

| | Example 11 | Example 12 | Example 13 | Example 14 |
|---|---|---|---|---|
| Nalmefene | 2.8 | 2.9 | 2.7 | 2.7 |
| hydrochloride | | | | |
| (calculated as | | | | |
| nalmefene) | | | | |
| Dodecylphosphocholine | 0.25 | 0.25 | 0.15 | 0.5 |
| n-Dodecyl-β-D-maltoside | NA | NA | NA | NA |
| Sodium chloride | 0.74 | 0.74 | 0.74 | 0.74 |
| Anhydrous citric acid | NA | NA | NA | NA |
| Sodium citrate | NA | NA | NA | NA |
| Hydrochloric | an | an | an | an |
| acid/Sodium hydroxide | appropriate | appropriate | appropriate | appropriate |
| | amount | amount | amount | amount |
| Benzalkonium chloride | 0.01 | 0.01 | 0.01 | 0.01 |
| Edetate disodium | 0.1 | 0.1 | 0.1 | 0.1 |
| Water | q.s. to 0.1 mL | q.s. to 0.1 mL | q.s. to 0.1 mL | q.s. to 0.1 mL |

### 1. Formulation and Preparation Process of Example 3

The formulation of Example 3 is shown in Table 1-3

TABLE 1-3

| Name | Single dose | Batch amount |
|---|---|---|
| Nalmefene hydrochloride | 3 mg (calculated as | 1.50 g (calculated as |
| | nalmefene) | nalmefene) |
| Dodecylphosphocholine | 0.25 mg | 0.125 g |
| Sodium chloride | 0.74 mg | 0.37 g |
| Edetate disodium | 0.1 mg | 0.05 g |
| Benzalkonium chloride | 0.01 mg | 0.005 g |
| Hydrochloric acid/sodium | an appropriate | an appropriate |
| hydroxide | amount | amount |
| Purified water | q.s. to 0.1 mL | q.s. to 50 mL |

### (1) Compounding

Preparation of 0.1 mol/L hydrochloric acid solution: 0.9 mL of hydrochloric acid was diluted to 100 mL with purified water, and mixed well.

An appropriate amount of purified water was measured into a beaker, a prescribed amount of each excipient was sequentially added and dissolved by stirring, and then a prescribed amount of nalmefene hydrochloride was added and dissolved by stirring. The pH value of the liquid was adjusted to 3.5-5.5 using hydrochloric acid/sodium hydroxide, and the volume was made up with purified water.

### (2) Filling

The drug solution was filled into a single-dose pharmaceutical spray pump (the spray pump includes: CONTAINER GLASS, PLUNGER, CONTAINER HOLDER, the supplier of the spray pump: Aptar Le Vaudreuil).

The preparation methods of Examples 1-2, 4-14, and Comparative Examples 1, 3, 4, 5, and 6 refer to Example 3.

### 2. Formulation and Preparation Process of Comparative Example 2

The formulation of Comparative Example 2 is as described in Table 1-4.

TABLE 1-4

| Name | Single dose | Batch amount |
|---|---|---|
| Nalmefene hydrochloride | 1.5 mg (calculated as | 0.30 g (calculated as |
| | nalmefene) | nalmefene) |
| Sodium chloride | 13.5 mg | 2.7 g |
| Hydrochloric acid | an appropriate amount | an appropriate amount |
| Water for injection | q.s. to 1.5 mL | q.s. to 300 mL |

### (1) Compounding

Water for injection was cooled to room temperature, and charged with nitrogen until the dissolved oxygen was ≤1 mg/L.

Preparation of 0.1 mol/L hydrochloric acid solution: 0.9 mL of hydrochloric acid was diluted to 100 mL with water for injection, and mixed well.

About 270 ml of water for injection was measured into a beaker, prescribed amounts of excipients were added and dissolved by stirring, and then a prescribed amount of nalmefene hydrochloride was added and dissolved by stirring. The pH of the drug solution was adjusted to 3.3±0.2, and the drug solution was made up to 300 mL. The drug solution was filtered through a 0.22 μm PES filter membrane. The drug solution was charged with nitrogen until the dissolved oxygen was ≤1 mg/L.

### (2) Filling and Capping

The drug solution was filled into glass vials with nitrogen purging (before filling) and then stoppering with nitrogen purging (after filling/before stoppering), then capped.

### (3) Sterilization

The filled samples were sterilized at 121° C. for 15 minutes.

### Experimental Example 1: Stability Investigation of Samples

(1) High-temperature test: The nasal spray preparations of nalmefene obtained in the Examples of the present invention were subjected to a high-temperature test at a temperature of 60° C.±2° C., and samples were collected on the 10th and 30th days for detection, respectively, to investigate the impurities and content data of the obtained nasal spray preparations. The results of Examples 1, 3, 4, 6, 7, 8 and 9, and Comparative Examples 4 and 7 are shown in Table 2-1.

(2) Acceleration test: The nasal spray preparations of nalmefene obtained in the Examples of the present invention were subjected to an acceleration test under the conditions of 40° C.±2° C./75% RH, and after one month of acceleration, samples were collected for detection, to investigate the impurities and content data of the obtained nasal spray preparations. The results of Examples 1, 3, 4, 6, 7, 8 and 9, and Comparative Examples 4 and 7 are shown in Table 2-1.

TABLE 2-1

| | Stability investigation results | | | | | |
|---|---|---|---|---|---|---|
| | | | | 60° C. ± | 60° C. ± | |
| | | | | 2° C - | 2° C. - | 1 month |
| | Detection point | | 0 day | 10 day | 30 day | (acceleration) |
| Example 1 | Impurity | P0Z3% | <0.05 | 0.29 | 0.64 | 0.10 |
| | | Total | <0.05 | 0.49 | 0.88 | 0.28 |
| | | impurities | | | | |
| | | (%) | | | | |
| | Assay (%) | | 100.9 | 100.7 | 98.0 | 99.5 |
| Example 3 | Impurity | P0Z3% | <0.05 | <0.05 | 0.09 | <0.05 |
| | | Total | <0.05 | 0.06 | 0.10 | <0.05 |
| | | impurities | | | | |
| | | (%) | | | | |
| | Assay (%) | | 103.8 | 101.7 | 102.2 | 102.1 |
| Example 4 | Impurity | P0Z3% | <0.05 | <0.05 | 0.10 | <0.05 |
| | | Total | <0.05 | 0.06 | 0.11 | 0.05 |
| | | impurities | | | | |
| | | (%) | | | | |
| | Assay (%) | | 100.8 | 100.6 | 100.2 | 100.0 |
| Example 6 | Impurity | P0Z3% | <0.05 | 0.10 | 0.26 | 0.07 |
| | | Total | 0.18 | 0.31 | 0.55 | 0.24 |
| | | impurities | | | | |
| | | (%) | | | | |
| | Assay (%) | | 103.3 | 101.4 | 102.3 | 101.9 |
| Example 7 | Impurity | P0Z3% | <0.05 | <0.05 | 0.08 | <0.05 |
| | | Total | <0.05 | <0.05 | 0.18 | <0.05 |
| | | impurities | | | | |
| | | (%) | | | | |
| | Assay (%) | | 103.8 | 101.2 | 102.3 | 102.0 |
| Example 8 | Impurity | P0Z3% | <0.05 | 0.06 | 0.13 | <0.05 |
| | | Total | <0.05 | 0.08 | 0.14 | <0.05 |
| | | impurities | | | | |
| | | (%) | | | | |
| | Assay (%) | | 100.8 | 101.5 | 101.7 | 100.9 |
| Example 9 | Impurity | P0Z3% | <0.05 | <0.05 | 0.10 | <0.05 |
| | | Total | <0.05 | <0.05 | 0.10 | <0.05 |
| | | impurities | | | | |
| | | (%) | | | | |
| | Assay (%) | | 102.5 | 103.5 | 103.9 | 102.9 |
| Comparative | Impurity | P0Z3% | <0.05 | 0.07 | 0.25 | 0.06 |
| Example 4 | | Total | <0.05 | 0.09 | 0.27 | 0.07 |
| | | impurities | | | | |
| | | (%) | | | | |
| | Assay (%) | | 101.1 | 101.8 | 102.1 | 102.3 |
| Comparative | Impurity | P0Z3% | 0.08 | NA | 0.26 | NA |
| Example 7 | | Total | 0.55 | NA | 0.82 | NA |
| | | impurities | | | | |
| | | (%) | | | | |

| | | | | |
|---|---|---|---|---|
| Assay (%) | 100.0 | NA | NA | NA |

Note:
NA indicates not yet investigated.

(3) High-temperature test: The nasal spray preparations of nalmefene obtained in the Examples of the present invention were subjected to a high-temperature test at a temperature of 60° C.±2° C., and samples were collected on the 5th, 10th, and 30th days for detection, respectively, to investigate the impurities and content data of the obtained nasal spray preparations. The results of Examples 10, 13, 14 and Comparative Example 5 are shown in Table 2-2.

(4) Acceleration test: The nasal spray preparations of nalmefene obtained in the Examples of the present invention were subjected to an acceleration test under the conditions of 40° C.±2° C./75% RH, and after one month of acceleration, samples were collected for detection, to investigate the impurities and content data of the obtained nasal spray preparations. The results of Examples 10, 13, 14 and Comparative Example 5 are shown in Table 2-2.

TABLE 2-2

| | Stability investigation results | | | | | | |
|---|---|---|---|---|---|---|---|
| | | | | 60° C. ± | 60° C.± | 60° C. ± | |
| | | | | 2° C. - | 2° C. - | 2° C. - | 1 month |
| | Detection point | | 0 day | 5 day | 10 day | 30 day | (acceleration) |
| Example 10 | Impurity | P0Z3% | <0.05 | 0.07 | 0.05 | 0.09 | <0.05 |
| | | Total | <0.05 | 0.07 | 0.05 | 0.09 | <0.05 |
| | | impurities | | | | | |
| | | (%) | | | | | |
| | Content (%) | | 102.2 | 101.5 | 102.6 | 104.3 | 103.4 |
| Example 13 | Impurity | P0Z3% | <0.05 | <0.05 | <0.05 | 0.14 | <0.05 |
| | | Total | <0.05 | <0.05 | <0.05 | 0.14 | <0.05 |
| | | impurities | | | | | |
| | | (%) | | | | | |
| | Content (%) | | 99.9 | 100.0 | 101.3 | 102.0 | 101.9 |
| Example 14 | Impurity | P0Z3% | <0.05 | <0.05 | <0.05 | 0.08 | <0.05 |
| | | Total | <0.05 | <0.05 | <0.05 | 0.08 | <0.05 |
| | | impurities | | | | | |
| | | (%) | | | | | |
| | Content (%) | | 100.9 | 101.0 | 101.0 | 102.1 | 100.6 |
| Comparative | Impurity | P0Z3% | <0.05 | NA | 0.14 | 0.22 | <0.05 |
| Example 5 | | Total | <0.05 | NA | 0.14 | 0.25 | <0.05 |
| | | impurities | | | | | |
| | | (%) | | | | | |

| | | | | | |
|---|---|---|---|---|---|
| Content (%) | 99.8 | NA | 102.3 | 101.6 | 101.0 |

Structural formula of related substance P0Z3:

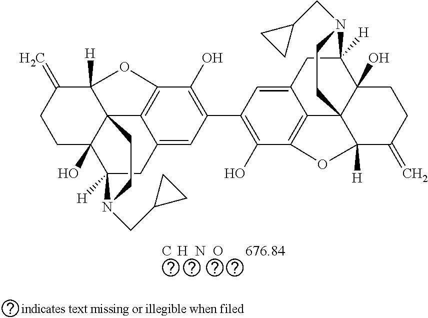

The data in Table 2-1 and Table 2-2 indicate that the sample stability of each example formulation meets the requirements.

The results of Examples 3, 4 and 9 show that the impurity level and high-temperature stability of the formulation of the present invention are superior to those of Comparative Example 7.

### Experimental Example 2: Pharmacokinetic Experiment

### 1. Experimental Animals

Beagle dogs (body weight of 12 kg-15 kg, supplier: Suzhou Xishan Zhongke Experimental Animal Co., Ltd., license number: SCXK(Su)2018-0001)

### 2. Experimental Apparatus and Detection Parameters

EDTA-K2 anticoagulant tube: brand Kangweishi (KWS)
Liquid chromatograph (Shimadzu, LC-30AD), mass spectrometer (SCIEX Triple Quad 4500)
LC-MS/MS detection conditions:
Instrument: UPLC-MS/MS (AB API 4500)
MS conditions: Electrospray ionization (ESI) source, positive ion detection

HPLC conditions: mobile phase A: 90% water+10% acetonitrile+0.1% formic acid+2 mM ammonium acetate; mobile phase B: 90% acetonitrile+10% water+0.1% formic acid+2 mM ammonium acetate; chromatographic column: Waters ACQUITY UPLC BEH C18 (2.1×50 mm, 1.7 μm); flow rate: 0.50 mL/min; chromatographic column temperature: 40° C.

| Time (minutes) | Mobile phase B (%) |
|---|---|
| 0.10 | 100 |
| 2.00 | 100 |
| 2.10 | 5 |
| 4.00 | Stop |

### 3. Experimental Method

Experimental method of nasal administration for Example 1: Four beagle dogs were administered with 1 dose of a nasal spray preparation of nalmefene hydrochloride by nasal spray respectively, and 2 mL of blood was collected from the forelimb vein before administration and at 2 min, 5 min, 10 min, 15 min, 30 min, 45 min, 1 h, 2 h, 4 h and 6 h after administration. The blood sample was placed in an EDTA-K2 anticoagulant tube, centrifuged at 4500 r/min for 10 min, and 50 μL of plasma was separated into a centrifuge tube. The remaining plasma was stored in a centrifuge tube for backup and frozen at −80° C. The content of nalmefene in the plasma was detected by LC-MS/MS.

The methods of nasal administration for Example 2 and Comparative Example 1 were the same as that for Example 1.

Experimental method of intramuscular injection for Comparative Example 2: Four beagle dogs were administered 1 dose of an injection of nalmefene hydrochloride (1.5 ml: 1.5 mg) intramuscularly respectively, and 2 mL of blood was collected from the forelimb vein before administration and at 2 min, 5 min, 10 min, 15 min, 30 min, 45 min, 1 h, 2 h, 4 h and 6 h after administration. The blood sample was placed in an EDTA-K2 anticoagulant tube, centrifuged at 4500 r/min for 10 min, and 50 μL of plasma was separated into a centrifuge tube. The remaining plasma was stored in a centrifuge tube for backup and frozen at −80° C. The content of nalmefene in the plasma was detected by LC-MS/MS.

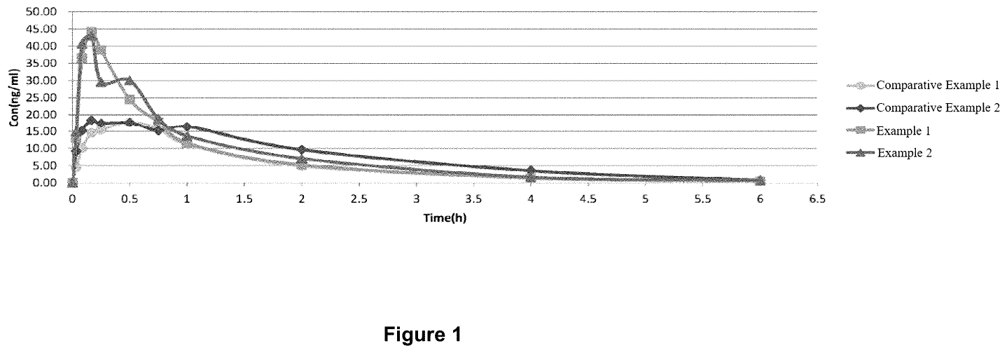

The main pharmacokinetic parameters of each experimental example are shown in Table 3, and the pharmacokinetic mean concentration-time curves of Examples 1-2 and Comparative Examples 1-2 are shown in FIG. 1 of the specification.

TABLE 3

| | Experimental Example | | | |
|---|---|---|---|---|
| | | | Comparative | Comparative |
| | Example 1 | Example 2 | Example 1 | Example 2 |
| Tₘₐₓ (h) | 0.27 | 0.27 | 0.42 | 0.56 |
| Cₘₐₓ (ng/mL) | 44.35 | 37.23 | 30.88 | 20.37 |
| AUCₗₐₛₜ | 41.64 | 37.32 | 42.61 | 46.17 |
| (h*ng/mL) | | | | |

The results in Table 3 and FIG. 1 of the specification show that the use of dodecylphosphocholine in the nasal spray preparation can significantly accelerate the absorption of nalmefene hydrochloride in the nasal cavity compared with not using dodecylphosphocholine, and the onset of action of the nasal spray preparation after administration is faster than intramuscular injection of 1.5 mg of the nalmefene hydrochloride injection.

### Experimental Example 3: Pharmacokinetic Experiment

### 1. Experimental Animals

Beagle dogs (body weight of 8 kg-10 kg, supplier: Chengdu Dossy Experimental Animal Co., Ltd., license number: SCXK(1)2019-031)

### 2. Experimental Apparatus and Detection Parameters

EDTA-K2 anticoagulant tube: brand Kangweishi (KWS)
Liquid chromatograph (Shimadzu, LC-30AD), mass spectrometer (SCIEX Triple Quad 4500)
LC-MS/MS detection conditions:
Instrument: UPLC-MS/MS (AB API 4500)
MS conditions: Electrospray ionization (ESI) source, positive ion detection

HPLC conditions: mobile phase A: 90% water+10% acetonitrile+0.1% formic acid+2 mM ammonium acetate; mobile phase B: 90% acetonitrile+10% water+0.1% formic acid+2 mM ammonium acetate; chromatographic column: Waters ACQUITY UPLC BEH C18 (2.1×50 mm, 1.7 μm); flow rate: 0.50 mL/min; chromatographic column temperature: 40° C.

| Time (minutes) | Mobile phase B (%) |
|---|---|
| 0.10 | 100 |
| 2.00 | 100 |
| 2.10 | 5 |
| 4.00 | Stop |

### 3. Experimental Method

Experimental method of nasal administration for Example 3: Four beagle dogs were administered with 1 dose of a nasal spray preparation of nalmefene hydrochloride by nasal spray respectively, and 2 mL of blood was collected from the forelimb vein before administration and at 2 min, 5 min, 10 min, 15 min, 30 min, 45 min, 1 h, 2 h, 4 h and 6 h after administration. The blood sample was placed in an EDTA-K2 anticoagulant tube, centrifuged at 4500 r/min for 10 min, and 50 μL of plasma was separated into a centrifuge tube. The remaining plasma was stored in a centrifuge tube for backup and frozen at −80° C. The content of nalmefene in the plasma was detected by LC-MS/MS.

The method of nasal administration for Comparative Example 4 was the same as that for Example 3.

The main pharmacokinetic parameters for each experimental example are shown in Table 4.

TABLE 4

| Experimental Example | Example 3 | Comparative Example 4 |
|---|---|---|
| Tₘₐₓ (h) | 0.21 | 0.38 |
| Cₘₐₓ (ng/mL) | 47.85 | 19.83 |
| AUCₗₐₛₜ (h*ng/mL) | 53.72 | 26.01 |

The results in Table 4 show that the use of dodecylphosphocholine can significantly accelerate the absorption of nalmefene hydrochloride in the nasal cavity. The results of Example 3 compared with Comparative Example 4 indicate that the absorption rate of nalmefene hydrochloride in Example 3 is faster, with Cmax and AUC values significantly higher than those in Comparative Example 4, and the effect of using dodecylphosphocholine to accelerate the absorption of nalmefene hydrochloride in the nasal cavity is superior to that of n-dodecyl-β-D-maltoside.

### Experimental Example 4: Pharmacokinetic Experiment

### 1. Experimental Animals

Beagle dogs (body weight of 8 kg-10 kg, supplier: Suzhou Xishan Zhongke Experimental Animal Co., Ltd. and Chengdu Dossy Experimental Animal Co., Ltd., license numbers: SCXK(Su)2018-0001 and SCXK(1)2019-031)

### 2. Experimental Apparatus and Detection Parameters

EDTA-K2 anticoagulant tube: brand Kangweishi (KWS)
Liquid chromatograph (Shimadzu, LC-30AD), mass spectrometer (SCIEX Triple Quad 4500)
LC-MS/MS detection conditions:
Instrument: UPLC-MS/MS (AB API 4500)
MS conditions: Electrospray ionization (ESI) source, positive ion detection

HPLC conditions: mobile phase A: 90% water+10% acetonitrile+0.1% formic acid+2 mM ammonium acetate; mobile phase B: 90% acetonitrile+10% water+0.1% formic acid+2 mM ammonium acetate; chromatographic column: Waters ACQUITY UPLC BEH C18 (2.1×50 mm, 1.7 μm); flow rate: 0.50 mL/min; chromatographic column temperature: 40° C.

| Time (minutes) | Mobile phase B (%) |
|---|---|
| 0.10 | 100 |
| 2.00 | 100 |
| 2.10 | 5 |
| 4.00 | Stop |

### 3. Experimental Method

Experimental method of nasal administration for Example 9: Four beagle dogs were administered with 1 dose of a nasal spray preparation of nalmefene hydrochloride by nasal spray respectively, and 2 mL of blood was collected from the forelimb vein before administration and at 2 min, 5 min, 10 min, 15 min, 30 min, 45 min, 1 h, 2 h, 4 h and 6 h after administration. The blood sample was placed in an EDTA-K2 anticoagulant tube, centrifuged at 4500 r/min for 10 min, and 50 μL of plasma was separated into a centrifuge tube. The remaining plasma was stored in a centrifuge tube for backup and frozen at −80° C. The content of nalmefene in the plasma was detected by LC-MS/MS.

The method of nasal administration for Comparative Example 5 was the same as that for Example 9.

The main pharmacokinetic parameters for each experimental example are shown in Table 5.

TABLE 5

| Experimental Example | Example 9 | Comparative Example 5 |
|---|---|---|
| Tₘₐₓ (h) | 0.22 | 0.25 |
| Cₘₐₓ (ng/mL) | 25.30 | 23.13 |
| AUCₗₐₛₜ (h*ng/mL) | 35.35 | 28.57 |

Table 5 shows that the use of dodecylphosphocholine can significantly accelerate the absorption of nalmefene hydrochloride in the nasal cavity. The absorption rate of nalmefene hydrochloride in Example 9 is faster, with AUC value significantly higher than that in Comparative Example 5, and the effect of using dodecylphosphocholine to accelerate the absorption of nalmefene hydrochloride in the nasal cavity is superior to that of n-dodecyl-3-D-maltoside.

The examples described above are merely descriptions of preferred embodiments of the present invention and are not intended to limit the scope of the present invention. Without departing from the spirit of the design of the present invention, various modifications and improvements made by the skilled in the art to the technical solutions of the present invention fall within the scope of protection determined by the claims of the present invention.

What is claimed is:

1. A pharmaceutical preparation for intranasal administration, characterized in that, the pharmaceutical preparation, in 50 μL-250 μL of an aqueous solution, comprises 1 mg-10 mg or 1 w/v %-10% (w/v) of nalmefene hydrochloride, calculated as nalmefene, or the hydrate thereof, and dodecylphosphocholine.

2. The pharmaceutical preparation according to claim 1, wherein the pharmaceutical preparation, in 50 μL-250 μL of an aqueous solution, comprises 2.5 mg-3.5 mg or 2.5%-3.5% (w/v) of nalmefene hydrochloride, calculated as nalmefene, or the hydrate thereof, and dodecylphosphocholine.

3. The pharmaceutical preparation according to claim 1, wherein the pharmaceutical preparation, in 50 μL-250 μL of an aqueous solution, comprises 2.7 mg-3.0 mg or 2.7%-3.0% (w/v) of nalmefene hydrochloride, calculated as nalmefene, or the hydrate thereof, and dodecylphosphocholine.

4. The pharmaceutical preparation according to claim 1, wherein the intranasal administration preparation is a nasal spray preparation.

5. The pharmaceutical preparation according to claim 1, wherein the pharmaceutical preparation comprises 0.15 mg-0.5 mg or 0.15%-0.5% (w/v) of dodecylphosphocholine.

6. The pharmaceutical preparation according to claim 5, wherein the pharmaceutical preparation comprises 0.15 mg-0.35 mg or 0.15%-0.35% (w/v) of dodecylphosphocholine.

7. The pharmaceutical preparation according to claim 6, wherein the pharmaceutical preparation comprises 3 mg of nalmefene hydrochloride, calculated as nalmefene, and 0.25 mg of dodecylphosphocholine; or
the pharmaceutical preparation comprises 3% (w/v) of nalmefene hydrochloride, calculated as nalmefene, and 0.25% (w/v) of dodecylphosphocholine.

8. The pharmaceutical preparation according to claim 6, wherein the pharmaceutical preparation comprises 2.7 mg of nalmefene hydrochloride, calculated as nalmefene, and 0.25 mg of dodecylphosphocholine; or
the pharmaceutical preparation comprises 2.7% (w/v) of nalmefene hydrochloride, calculated as nalmefene, and 0.25% (w/v) of dodecylphosphocholine.

9. The pharmaceutical preparation according to claim 1, wherein the pharmaceutical preparation further comprises a pH adjusting agent or a buffer to achieve a pH of 3.5-5.5; and/or
the pharmaceutical preparation further comprises an osmotic pressure regulator to achieve an osmotic pressure ratio of 0.2-2.

10. The pharmaceutical preparation according to claim 1, wherein the pharmaceutical preparation further comprises 0.001 mg-1 mg or 0.001%-1% (w/v) of a preservative.

11. The pharmaceutical preparation according to claim 1, wherein the pharmaceutical preparation further comprises 0.001 mg-1 mg or 0.001%-1% (w/v) of a stabilizer.

12. The pharmaceutical preparation according to claim 1, wherein the pharmaceutical preparation comprises, in 100 μL of an aqueous solution, 3 mg of nalmefene hydrochloride, calculated as nalmefene, and 0.25 mg of dodecylphosphocholine; or
the pharmaceutical preparation comprises, in 100 μL of an aqueous solution, 3% (w/v) of nalmefene hydrochloride, calculated as nalmefene, and 0.25% (w/v) of dodecylphosphocholine.

13. The pharmaceutical preparation according to claim 1, wherein the pharmaceutical preparation, in 100 μL of an aqueous solution, comprises 3 mg of nalmefene hydrochloride, calculated as nalmefene, 0.15 mg-0.5 mg of dodecylphosphocholine, 0.74 mg of sodium chloride, and hydrochloric acid and/or sodium hydroxide to achieve a pH of 3.5-5.5; or
the pharmaceutical preparation, in 100 μL of an aqueous solution, comprises 3% (w/v) of nalmefene hydrochloride, calculated as nalmefene, 0.15%-0.5% (w/v) of dodecylphosphocholine, 0.74% (w/v) of sodium chloride, and hydrochloric acid and/or sodium hydroxide to achieve a pH of 3.5-5.5.

14. The pharmaceutical preparation according to claim 1, wherein the pharmaceutical preparation, in 100 μL of an aqueous solution, comprises 3 mg of nalmefene hydrochloride, calculated as nalmefene, 0.15 mg-0.35 mg of dodecylphosphocholine, 0.74 mg of sodium chloride, 0.1 mg of edetate disodium, 0.01 mg of benzalkonium chloride, and hydrochloric acid and/or sodium hydroxide to achieve a pH of 3.5-5.5; or
the pharmaceutical preparation, in 100 μL of an aqueous solution, comprises 3% (w/v) of nalmefene hydrochloride, calculated as nalmefene, 0.15%-0.35% (w/v) of dodecylphosphocholine, 0.74% (w/v) of sodium chloride, 0.1% (w/v) of edetate disodium, 0.01% (w/v) of benzalkonium chloride, and hydrochloric acid and/or sodium hydroxide to achieve a pH of 3.5-5.5.

15. The pharmaceutical preparation according to claim 1, wherein the pharmaceutical preparation, in 100 μL of an aqueous solution, comprises 2.7 mg of nalmefene hydrochloride, calculated as nalmefene, and 0.25 mg of dodecylphosphocholine; or
the pharmaceutical preparation, in 100 μL of an aqueous solution, comprises 2.7% (w/v) of nalmefene hydrochloride, calculated as nalmefene, and 0.25% (w/v) of dodecylphosphocholine.

16. The pharmaceutical preparation according to claim 1, wherein the pharmaceutical preparation, in 100 μL of an aqueous solution, comprises 2.7 mg of nalmefene hydrochloride, calculated as nalmefene, 0.15 mg-0.5 mg of dodecylphosphocholine, 0.74 mg of sodium chloride, and hydrochloric acid and/or sodium hydroxide to achieve a pH of 3.5-5.5; or
the pharmaceutical preparation, in 100 μL of an aqueous solution, comprises 2.7% (w/v) of nalmefene hydrochloride, calculated as nalmefene, 0.15%-0.5% (w/v) of dodecylphosphocholine, 0.74% (w/v) of sodium chloride, and hydrochloric acid and/or sodium hydroxide to achieve a pH of 3.5-5.5.

17. The pharmaceutical preparation according to claim 1, wherein the pharmaceutical preparation, in 100 μL of an aqueous solution, comprises 2.7 mg of nalmefene hydrochloride, calculated as nalmefene, 0.15 mg-0.35 mg of dodecylphosphocholine, 0.74 mg of sodium chloride, and hydrochloric acid and/or sodium hydroxide to achieve a pH of 3.5-5.5; or
the pharmaceutical preparation, in 100 μL of an aqueous solution, comprises 2.7% (w/v) of nalmefene hydrochloride, calculated as nalmefene, 0.15%-0.35% (w/v) of dodecylphosphocholine, 0.74% (w/v) of sodium chloride, and hydrochloric acid and/or sodium hydroxide to achieve a pH of 3.5-5.5.

18. The pharmaceutical preparation according to claim 1, wherein the pharmaceutical preparation, in 100 μL of an aqueous solution, comprises 2.7 mg of nalmefene hydrochloride, calculated as nalmefene, 0.15 mg-0.35 mg of dodecylphosphocholine, 0.74 mg of sodium chloride, 0.1 mg of edetate disodium, 0.01 mg of benzalkonium chloride, and hydrochloric acid and/or sodium hydroxide to achieve a pH of 3.5-5.5; or
the pharmaceutical preparation, in 100 μL of an aqueous solution, comprises 2.7% (w/v) of nalmefene hydrochloride, calculated as nalmefene, 0.15%-0.35% (w/v) of dodecylphosphocholine, 0.74% (w/v) of sodium chloride, 0.1% (w/v) of edetate disodium, 0.01% (w/v) of benzalkonium chloride, and hydrochloric acid and/or sodium hydroxide to achieve a pH of 3.5-5.5.

19. The pharmaceutical preparation according to claim 1, wherein the pharmaceutical preparation, in 100 μL of an aqueous solution, comprises 2.7 mg of nalmefene hydrochloride, calculated as nalmefene, 0.25 mg of dodecylphosphocholine, 0.74 mg of sodium chloride, 0.1 mg of edetate disodium, 0.01 mg of benzalkonium chloride, and hydrochloric acid and/or sodium hydroxide to achieve a pH of 3.5-5.5; or
the pharmaceutical preparation, in 100 μL of an aqueous solution, comprises 2.7% (w/v) of nalmefene hydrochloride, calculated as nalmefene, 0.25% (w/v) of dodecylphosphocholine, 0.74% (w/v) of sodium chloride, 0.1% (w/v) of edetate disodium, 0.01% (w/v) of benzalkonium chloride, and hydrochloric acid and/or sodium hydroxide to achieve a pH of 3.5-5.5.

20. A method of preventing or treating opioid overdose or the symptoms thereof in a patient, comprising
administering to the patient the pharmaceutical preparation according to claim 1.
